(19) United States
(12) Patent Application Publication
(10) Pub. No.: US 2009/0326412 A1
(43) Pub. Date: Dec. 31, 2009

(54) Biopsy Device with Rotating Needle

(76) Inventor: Robert L. Pakter, Tucson, AZ (US)

Correspondence Address: FISH & RICHARDSON P.C., PO BOX 1022, MINNEAPOLIS, MN, 55440-1022, US

(21) Appl. No.: 12/472,322

(22) Filed: May 26, 2009

Related U.S. Application Data
(60) Provisional application No. 61/128,740, filed on May 23, 2008.

(51) Int. Cl. A61B 10/02

(52) U.S. Cl. 600/567

(57) ABSTRACT

A biopsy device configured to rotate a needle about a longitudinal axis when resecting tissue, and a method of performing a biopsy using the same. Also disclosed is a needle configured to resect tissue by a rotational motion, for example, when operating in conjunction with the device herein.

### CLAIM OF PRIORITY

This application claims the benefit of priority to U.S. Provisional Application No. 61/128,740, filed May 23, 2008, the contents of which are incorporated herein by reference in their entirety.

### TECHNICAL FIELD

This application relates to a biopsy device, more particularly to a biopsy device configured to rotate a needle about a longitudinal axis when resecting tissue; the application also relates to a needle configured to resect tissue by a rotational motion.

### BACKGROUND

In the practice of medical biopsy, samples obtained may utilize either the so called fine needle aspiration (FNA) technique or the core biopsy technique. In FNA, a semi-liquid specimen is aspirated, usually through a small gauge needle (for example 22 g to 25 g) and subsequently expressed onto slides for examination of individual cells, or into a container with fixative for centrifugation and later cytologic analysis. By contrast, core biopsy typically utilizes larger gauge needles (for example 14g to 18 g) to yield a cohesive specimen which is placed in a fixative agent and later sliced, stained and microscopically examined for histologic analysis.

Often regions of tissue of interest may be quite small, for example a lesion 5 mm or less in one dimension. Such lesions may not be observable to the naked eye and are demonstrated only with the use of an imaging modality (such as ultrasound, computed tomography, or magnetic resonance imaging). Given the small size of such lesions it is of critical importance that the biopsy device samples only the region of interest in the immediate proximity of the lesion.

### SUMMARY

In general, in one aspect of the technology described herein includes a biopsy device configured to perform a biopsy by rotating a needle.

The technology described herein further includes a biopsy device comprising: a proximal casing having a longitudinal axis; and a distal needle as further described herein affixed to the casing, and configured to rotate about the longitudinal axis.

In another aspect, the technology includes a biopsy device comprising: a casing having a proximal end and a distal end, and defining a longitudinal axis running between the proximal and distal ends; a first chamber disposed within the casing and opening to the distal end; a second chamber disposed within the casing between the first chamber and the proximal end; a hub disposed coaxially within the first chamber and including a feature on its external surface; a needle assembly mounted on and disposed coaxially with the hub, the needle having a lumen, the lumen in fluid connection with the second chamber; and a control configured to engage the feature on the external surface of the hub, and to cause a rotation of the hub and needle assembly about the longitudinal axis.

The technology herein also includes a method of performing a biopsy of a tissue, the method comprising: controlling a device as further described herein so that the needle is inserted into the tissue; rotating the needle about the longitudinal axis of the device, thereby cutting tissue; reducing pressure in the lumen; and withdrawing the needle from the tissue, thereby removing a portion of cut tissue.

The technology further includes a biopsy needle comprising: a tubular body having a centrally disposed longitudinal axis running between distal and proximal ends of the body, the body further comprising a lumen disposed along the longitudinal axis, and a distal opening; and one or more elongated members extending distally from the distal opening and contiguous with the tubular body, each of the one or more elongated members having a first edge and an opposed second edge, the first edge configured to perform a cutting action as the needle is rotated about the longitudinal axis in a first direction, the second edge optionally configured to perform a cutting action as the needle is rotated about the longitudinal axis in a second direction, the second direction being in an opposite rotational sense to the first direction.

Certain embodiments may have one or more of the following advantages. The device, needle, and rotational cutting method described herein can prevent sampling of adjacent non-target tissue such that tissue sampled is precisely from the locus of interest.

The details of one or more embodiments are set forth in the accompanying drawings and the description herein. Other features, aspects, and advantages will become apparent from the description, the drawings, and the claims.

### BRIEF DESCRIPTION OF THE DRAWINGS

FIG. 1 is a longitudinal cross-sectional view of one embodiment of a biopsy device as further described herein.

FIG. 2A is a longitudinal cross-sectional view of a hub isolated from the device shown in FIG. 1.

FIG. 2B is a plan view of the hub shown in FIG. 2A.

FIG. 2C is a transverse cross-sectional view of the hub shown in FIGS. 2A-B.

FIG. 3A is a longitudinal cross-sectional view of a casing of the biopsy device shown in FIG. 1.

FIG. 3B is a elevation view of the casing shown in FIG. 3A.

FIG. 3C is a transverse cross-sectional view of a distal region of the casing shown in FIGS. 3A-B, and a control.

FIG. 3D is a transverse cross-sectional view of a proximal region of the casing shown in FIGS. 3A-B.

FIG. 3E shows an exemplary control disassembled from the device.

FIGS. 4A-B are cross-sectional views of a diaphragm assembly isolated from the device shown in FIG. 1.

FIG. 5 is a longitudinal cross-sectional view of an end cap isolated from the device shown in FIG. 1.

FIGS. 6A-N are cross-sectional and perspective views of various embodiments of a needle for rotating while carrying out a biopsy.

FIGS. 7A-B are cross-sectional views of the device shown in FIG. 1, illustrating movement of the control and hub, and movement of the diaphragm assembly.

FIGS. 7C-D are transverse cross-sectional views of a proximal region of the casing and diaphragm assembly shown in FIGS. 7A-B, illustrating a ratcheting mechanism of the second chamber.

FIG. 8A is a longitudinal cross-sectional view of an alternative embodiment of an device as further described herein.

FIG. 8B is a transverse cross-sectional views of a proximal region of the casing and diaphragm assembly of the embodiment shown in FIG. 8A.

FIG. 9 is a longitudinal cross-sectional view of another embodiment of an device.

The figures represent only certain embodiments of the disclosure. They are not necessarily drawn to scale, and may emphasize or exaggerate certain features to facilitate illustration and explanation. Like reference numbers in the drawings refer to like parts.

### DETAILED DESCRIPTION

### Biopsy Device

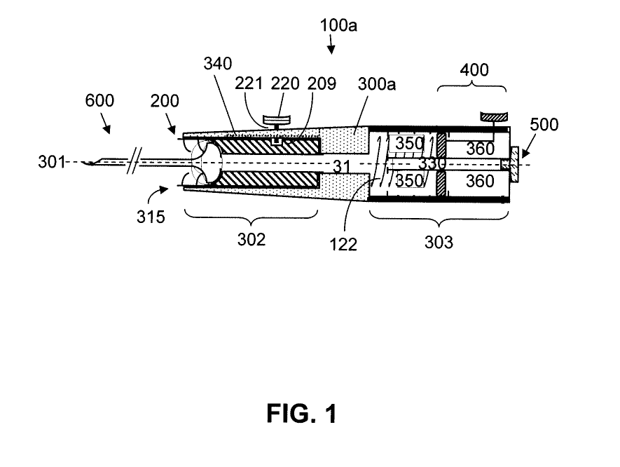

FIG. 1 generally illustrates, in cross-sectional view, principal features of an exemplary embodiment of a biopsy device 100a. Biopsy device 100a comprises hub 200, casing 300a, diaphragm assembly 400, and end cap 500. A first chamber 315 is disposed at distal end 302 of casing 300a, and includes a distal opening of the device. A longitudinal axis 301 runs centrally through the length of the device. The axis passes through the centroid of a transverse cross-section of the body at each point along its length. Hub 200 is disposed coaxially within receiving chamber 315 and encloses a lumen. Hub 200 is shown as fitting snugly within chamber 315, though this need not be the case. Hub 200 is shown with a helical groove inscribed on its outer surface, though other surface features are possible.

A control is configured to engage with hub 200 and to cause a rotational motion of hub 200 about the longitudinal axis. In the exemplary embodiment of FIG. 1, a distal slot 340 is disposed at distal end 302 of casing 300a and runs parallel to the longitudinal axis. A handle 220, is connected to stem 221, which slidably inserts through distal slot 340 and engages hub 200 at helical groove 209.

Casing 300a also includes a barrel portion 310 that connects first chamber 315 and a second chamber, at proximal end 303 of the casing 300a. The second chamber is divided into a proximal portion 350 and a distal portion 360. A channel 330 is also disposed at proximal end 303. In certain embodiments, distal portion of the second chamber 350 and proximal portion of second chamber 360 may surround channel 330, as shown. In some embodiments, a spring 122 may be included in the distal portion of the second chamber. Diaphragm assembly 400, in its various parts and as explained further herein, is disposed within second chamber 350, within casing 300a, and at the exterior of casing 300a. End cap 500 is reversibly inserted into channel 330.

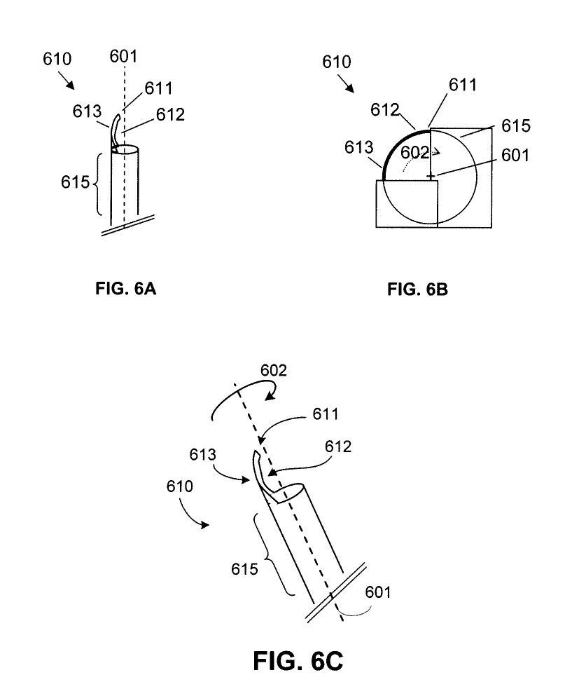

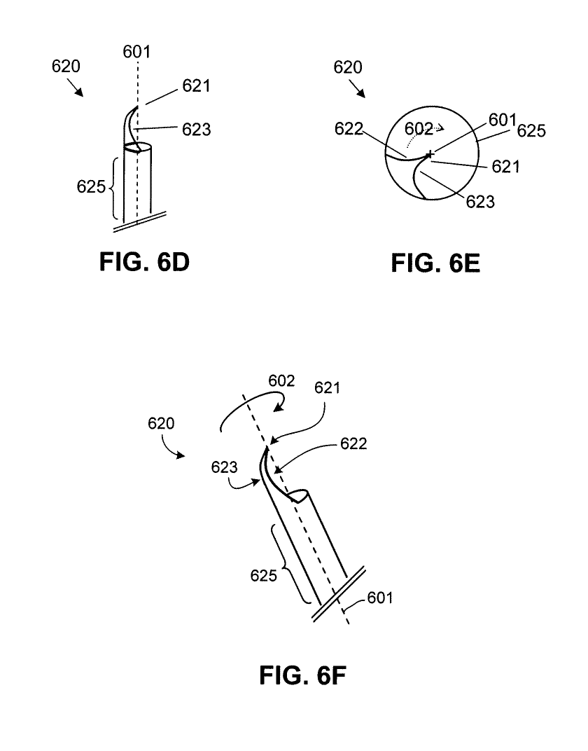

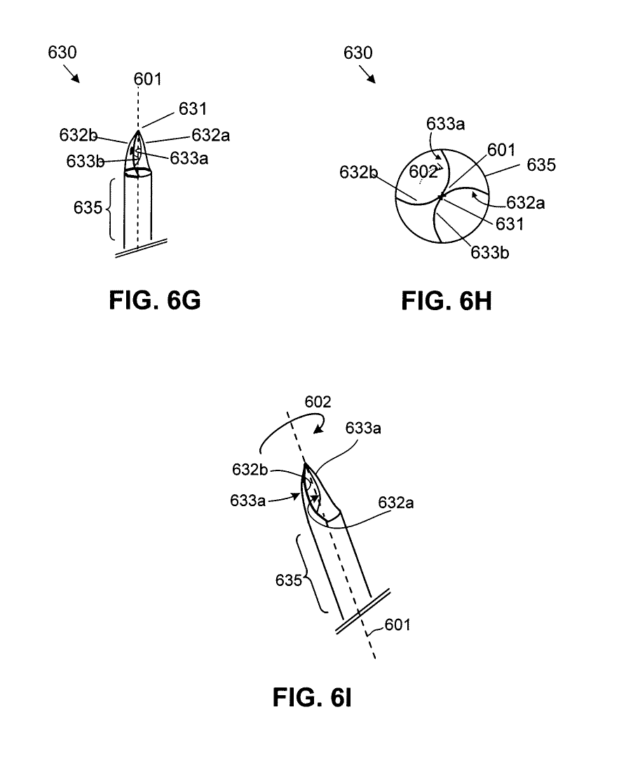

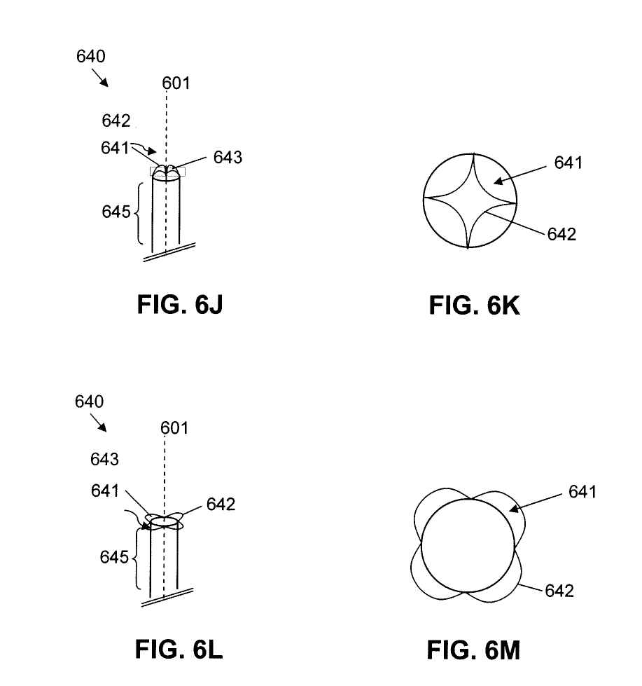

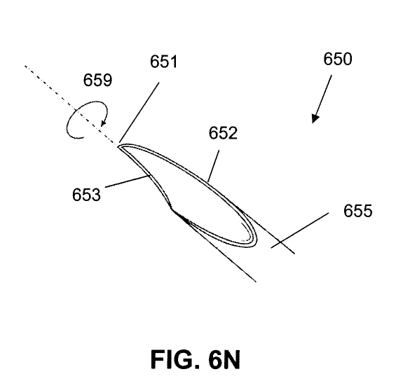

Needle assembly 600, comprising a needle and a connecting member that attaches the needle to hub 200, may be reversibly attached to hub 200 (e.g., via a luer connection) as shown, and is disposed coaxially with longitudinal axis 301. A lumen of needle 600 can align with barrel portion 310 so as to create a continuous channel through device 100a via the lumen of hub 200. As referred to throughout this application, needle 600 may be a conventional biopsy needle, including those used for both fine biopsies and core biopsies, or any of needles 610, 620, or 630 illustrated in FIGS. 6A-M, as further described herein. Other needles may also be used.

As described further herein, the operation of exemplary device 100a is as follows. During use (e.g., after needle 600 is attached to the device and inserted into a tissue of interest in a subject), an operator may move control 220 back and forth along slot 340 (e.g., in the proximal and distal directions), engaging helical groove 209 with stem 221. As a result, the translational motion of the control is converted into rotational motion of hub 200 so that the hub and attached needle assembly 600 will rotate around longitudinal axis 301. Such rotation can be effected to cut tissue in a precise manner and with single-hand operation of the device, freeing the other hand, for example to control other instrumentation, such as an ultrasound probe, or another device.

Other manners of causing a rotational motion of the needle and hub can be envisaged and are within the scope of the present description. For example, the hub may have a series of parallel circumferential grooves on its outer surface, or a set of teeth, that may be held directly by an operator and turned manually via a point of access.

A user may also apply suction by pulling back (e.g. in the proximal direction) on diaphragm assembly 400. Optionally, a spring 122 can be used to facilitate the proximal translation of diaphragm assembly 400. Proximal movement of assembly 400 applies suction upon the fluid in barrel portion 310, channel 330, and second chamber 350 and through needle 600 to create suction on the target tissue. Subsequently, the needle may be removed from the patient and the sample obtained may be extruded from the device, again with controlled, one-hand operation.

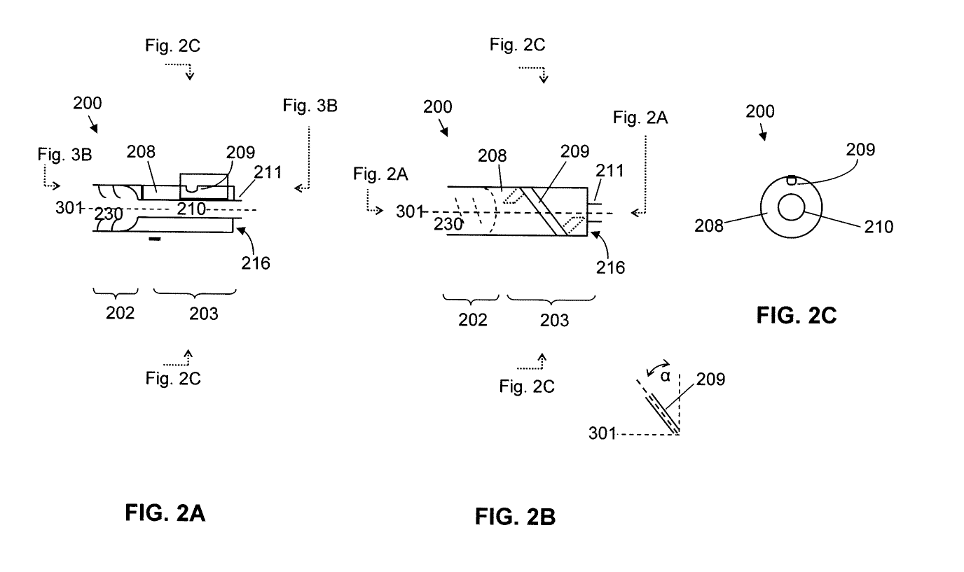

FIGS. 2-5 illustrate individual components of the exemplary biopsy device of FIG. 1. For clarity, shading of the various components has been removed in some illustrations.

### Hub and Control of Rotational Motion

FIG. 2A illustrates hub 200 in the same cross-sectional view as shown in FIG. 1. Hub 200 includes, at its distal end 202, needle mount 230 which is sized and configured (e.g., with a luer-lock fitting) so that a needle may be reversibly attached to it. Hub 200 includes a housing 208, which defines helical groove 209 as well as lumen 210. Lumen 210 may be optionally extended past housing 208 (toward proximal end 203) by a stem 211. The optional proximal stem 211 may insert into channel 330 on proximal portion of device 303 thereby increasing the effective lumen length 210 and limiting spillage of the aspirated content into the distal portion of the second chamber 350. Should such spillage occur, however, it is retrievable by reciprocal motion of assembly 400 as detailed elsewhere herein.

FIG. 2B shows hub 200 in an elevated view. In this view, needle mount 230 (shown in dashed lines) is occluded by housing 208. Helical groove 209 (of which a portion is shown) extends around the perimeter of needle hub housing 208. Helical groove 209 is canted at angle α with respect to a plane perpendicular to axis 301. Angle α may be selected to facilitate rotation of the hub during operation of the device. For example, angle α may be between approximately 5 degrees and approximately 60 degrees, such as between approximately 15 and approximately 30 degrees. From its beginning to its end, helical groove 209 may extend partially or completely around housing 208 and around longitudinal axis 301, depending on the length of housing 208 and the orientation of helical groove 209. The extent to which helical groove 209 extends around housing 208 determines the amount of rotation of the hub as control 220 is moved proximally or distally. Helical groove 209 may extend, in various embodiments, approximately 360° about axis 301. In some embodiments, helical groove 209 may extend up to or less than approximately 360° around longitudinal axis 301, e.g., between approximately 90° and approximately 360°. In other embodiments, it may extend more than approximately 360° around longitudinal axis 301, e.g., between approximately 360° and approximately 540°, and may encompass multiple, such as 2, 3, 4, 5, 6, 8, 10, 12, 15, 16, or 20 revolutions around 301. Helical groove 209 may extend in either a clockwise or counterclockwise direction when viewed down the longitudinal axis in a distal direction.

FIG. 2C illustrates a transverse cross-sectional view of hub 200 from FIG. 1. In this view, a cross section of helical groove 209 can be seen. In the embodiment shown, helical groove 209 has a rounded (e.g. “u” shaped) cross section (e.g., in order to decrease friction between helical groove 209 and stem 221, not shown here), but it may take other shapes in other embodiments. As shown in this embodiment, lumen 210 and needle hub 208 have circular cross sections, and lumen 210 is concentric with needle hub 208.

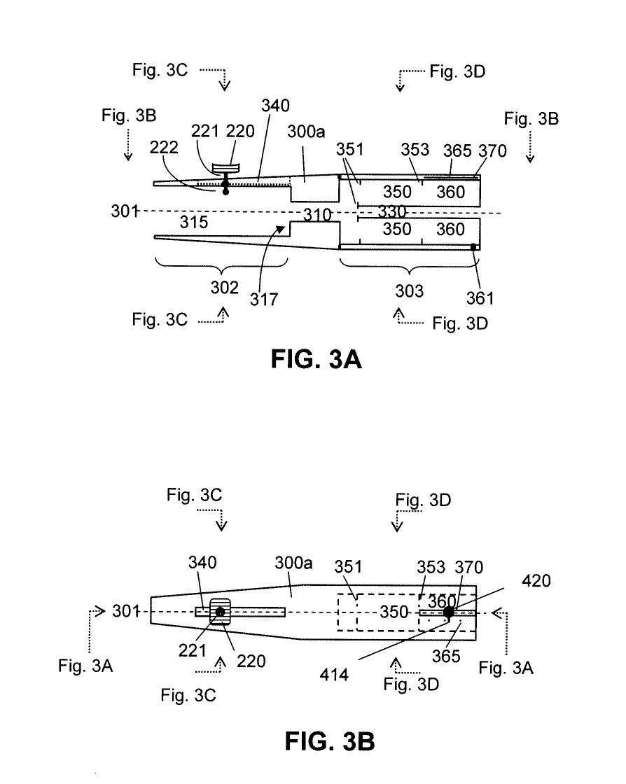

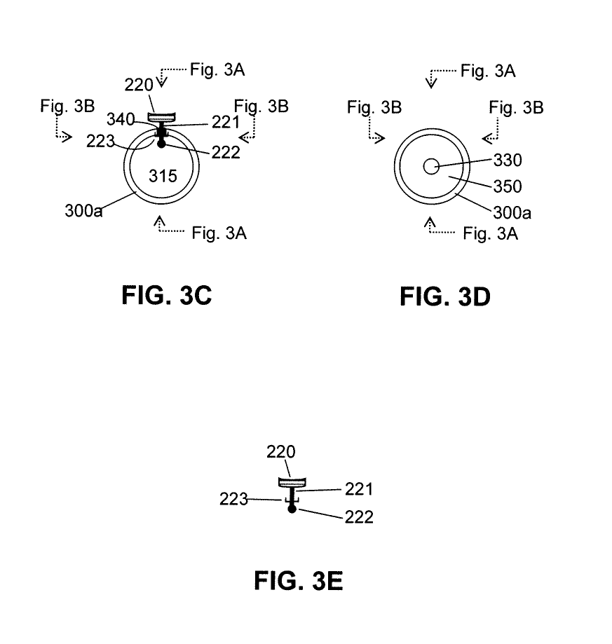

FIG. 3A illustrates casing 300a in a longitudinal cross-sectional view through its center. Disposed at distal end 302 is first chamber 315, which is configured to receive hub 200 (not shown). Leading from first chamber 315 toward the proximal end, 303, is a center portion 310, referred to also as a barrel portion 310, which separates the first chamber from the second chamber and contains a lumen that, continuing in the proximal direction, leads to second chamber 350 and channel 330. The distal portion of second chamber 350 and channel 330 are in fluid communication with one another, and with the lumen in barrel portion 310. When hub 200 is attached to the device, barrel portion 310 is in fluid communication with lumen 210. When a needle is attached to the device, lumen 210 is in fluid communication with the lumen of the needle, so that the distal portion of the second chamber 350 is in fluid communication with the lumen of the needle.

Referring still to FIG. 3A, distal slot 340 is disposed at the distal end 302 of casing 300a, and configured to allow stem 221 to slidably move therein. In this embodiment of the device, end 222 of stem 221 engages helical groove 209 of hub 200, as described elsewhere herein.

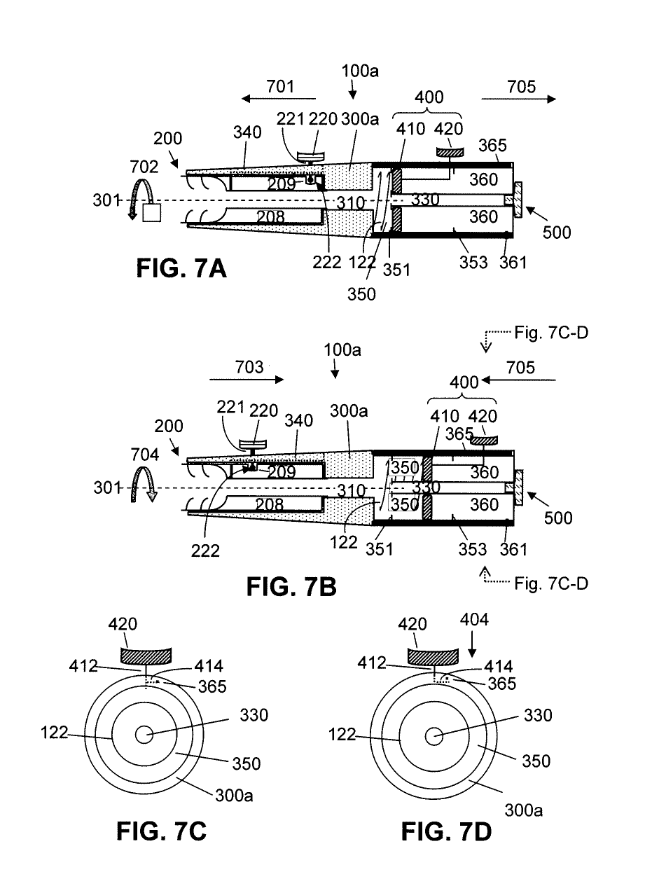

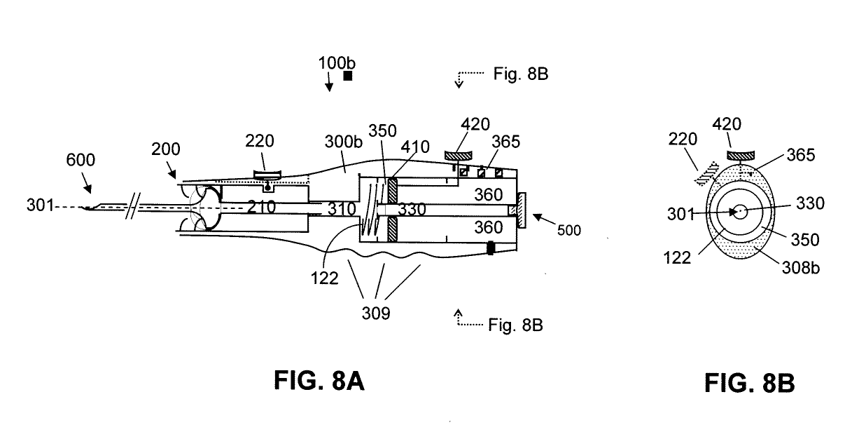

Referring still to FIG. 3A, proximal slot 370 is disposed at the proximal end 303 of casing 300a, and configured to allow part of the diaphragm assembly 400 (not shown) to slidably move within distal portion of the second chamber. Distal portion of second chamber 350 includes distal end-stops 351, and proximal end-stops 353. As explained elsewhere herein, these end-stops provide distal and proximal endpoints for the slidable movement of diaphragm assembly 400 in the distal and proximal directions, respectively. In some embodiments, the proximal portion of second chamber 360 also includes a ratcheting mechanism for providing intermediate stopping points for the slidable movement of diaphragm 400, via a control member. For example, in the embodiment illustrated (see also FIG. 8A), intermediate stops 365 are disposed within casing 300a. These intermediate stops may be, e.g., perforations, or other types of depressions or recessions in casing 308a, and disposed alongside proximal groove 370. As described elsewhere herein (e.g., FIG. 7D), these intermediate stops may engage an arm 414 on the diaphragm assembly, enabling a user to temporarily and controllably interrupt the slidable movement of the diaphragm assembly 400 between its distal and proximal endpoints. While the illustration in FIG. 3B shows three intermediate stops, casing 300a may contain any number of intermediate stops consistent with the construction and operation of this device. Note also that other ways for providing a ratcheting effect to diaphragm assembly 400 can be envisaged, for example, the inner surface of the proximal portion of the second chamber 360 may contain bumps or ridges or be rough, thereby providing a friction-based way to interrupt the movement of the diaphragm assembly by engagement of arm 414.

Referring still to FIG. 3A, in certain embodiments, the proximal portion of the second chamber 360 may include a vent, such as vent 361. As described elsewhere herein, vent 361 allows air to pass in and out of the proximal portion of the second chamber 360 to maintain atmospheric pressure when diaphragm assembly 400 is moved in the distal or proximal directions.

FIG. 3B shows an elevation view of casing 300a. Visible in this view is distal slot 340, at distal end 302, extending parallel to longitudinal axis 301. Proximal slot 370, at proximal end 303, (accommodating parts of diaphragm assembly 400, as discussed herein) also extends parallel to longitudinal axis 301. Also in this view are intermediate stops 365, disposed laterally from proximal slot 370 and which are engaged by side arm 414. Other components, corresponding to those shown in FIG. 3A, may be shown in dashed lines, indicating they are occluded by casing 300a in this view.

FIG. 3C illustrates a transverse cross-section of a distal region of casing 300a, as indicated in FIGS. 3A-B. This part of casing 300a is shown here to have a circular cross section, but in other embodiments may have a cross section of a different shape (e.g., elliptical). Casing 300a encloses receiving chamber 315, which generally will have a circular cross section, as shown, to enable smooth rotation of hub 200 within it. Control handle 220 is connected to stem 221, which inserts through distal slot 340 to engage in a helical groove on an external surface of hub 200 (not shown). In some embodiments, stem 221 may be slidably connected to casing 300a, for example, by bars 223 (see also FIG. 3E) which engage rails (not shown) in casing 300a along the sides of distal slot 340. In another embodiment, stem tip 222 may represent a ball bearing fabricated of a metal such as stainless steel or plastic such as polycarbonate. Distal slot 340 and stem tip 222 may be covered with a lubricious substance such as silicone or polytetrafluoroethylene to facilitate movement. Other means of connecting the control to the casing of the device may be envisaged.

FIG. 3D illustrates a transverse cross-section of a proximal region of casing 300a, as indicated in FIGS. 3A-B. Casing 300a, second chamber 350, and channel 330 are shown in this implementation to have circular cross-sections, and are disposed concentrically around a central axis (longitudinal axis 301, not shown). In other embodiments, they may have other cross-sectional shapes and configurations. As also shown in this embodiment, the distal portion of second chamber 350 surrounds channel 330. Other arrangements can be envisaged, however. For example, the second chamber may be disposed only on one side of the channel.

FIG. 3E shows a cross-sectional view shown in FIG. 3C of control 220 isolated from casing 300a, for clarity.

### Diaphragm Assembly

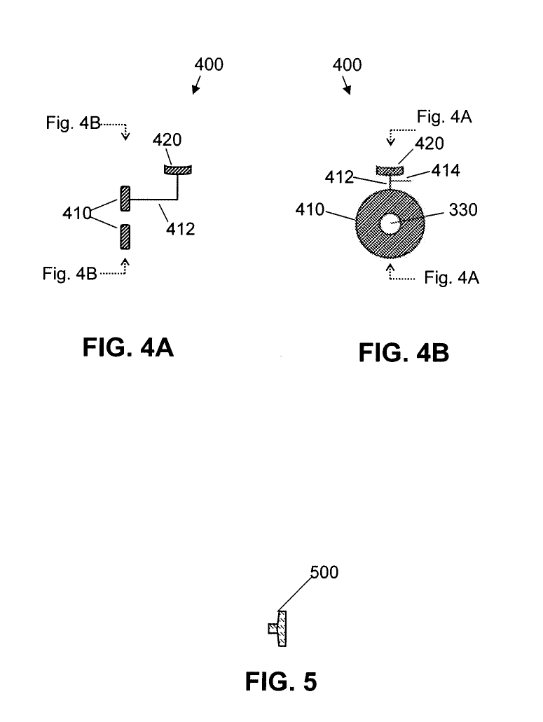

FIGS. 4A-B illustrate two views of an exemplary diaphragm assembly 400, comprising a partition 410, a rod 412, and a handle 420. FIG. 4A, viewed from the same cross-sectional view as shown in FIG. 1, shows two regions of partition 410. In the exemplary device shown, partition 410 is a disc that forms an airtight seal with the walls of the distal portion of the second chamber 350. Partition 410 is configured to move along the longitudinal axis within the distal portion of the second chamber 350 so that a volume of the distal portion of the second chamber distal to the partition is in fluid communication with lumen 310. Partition 410 is connected to handle 420 via flexible rod 412. Flexible rod 412 is configured so that in the device, part of flexible rod 412 slidably inserts through casing 300a, such as through distal slot 370. (See FIGS. 3A-B.) Flexible rod is typically made from a sprung material that permits a user to reversibly deflect it. Since flexible rod 412 extends within the proximal portion of the second chamber 360 only to the proximal wall of partition 410, and since the slot in casing 300a through which the rod extends does not overlap with the distal portion of the second chamber, the integrity of the vacuum or partial vacuum within barrel portion 310 and the distal portion of second chamber 350 is never compromised. This allows a vacuum or positive pressure to be generated, which might otherwise be compromised if groove 370 lay distal to the distal surface of partition 410. In the exemplary embodiment of FIGS. 1 and 3A-B, member 412 extends proximal to partition 410 and travels in groove 370 which lies proximal to partition 410 at all positions of 410.

FIG. 4B shows diaphragm assembly 400 and its component parts in transverse cross-section. Bar 414 extends laterally from flexible rod 412. Bar 414 may engage intermediate stops 365 (see description of FIG. 3A, and elsewhere herein). Bar 414 represents one way to arrest proximal or distal motion of the partition: by catching a member such as bar 414 on a lug or protrusion or indentation on the interior of the casing. In another embodiment, flexible rod travels longitudinally in a slot in the casing but the slot additionally has lateral, or transverse, notches in it, such that by a sideways motion of handle 420, the connector between handle 420 and rod 412 engages in a notch thereby arresting proximal or distal motion of the diaphragm assembly.

FIG. 5 shows end cap 500. End cap 500 may be inserted (e.g., by friction fit, luer screw connection, etc.) into channel 330 (see FIGS. 1 and 3). End cap 500 may be solid, or may have an internal lumen allowing a rod such as a stylet, tube, or other instruments to be inserted through it and into the second chamber to assist in the controlled extrusion of contents aspirated into lumens 210, 310 and 330. When inserted into channel 330, end cap 500 will form an air-tight seal with the wall of the chamber. As noted elsewhere herein, channel 330 is in fluid communication with distal portion of second chamber 350, and channel 310. A syringe (not shown) containing fluid such as fixative or saline may be connected in lieu of cap 500 and used to evacuate the lumens as well as any contents which may have accumulated in chamber 550.

### Needle

The technology herein includes a biopsy needle comprising: a tubular body having a centrally disposed longitudinal axis running between distal and proximal ends of the body, the body further comprising a lumen disposed along the longitudinal axis, and a distal opening; and one or more, such as two, elongated members extending distally from the distal opening and contiguous with the tubular body, each of the one or more elongated members having a first edge and an opposed second edge, the first edge configured to perform a cutting action as the needle is rotated about the longitudinal axis in a first direction, the second edge optionally configured to perform a cutting action as the needle is rotated about the longitudinal axis in a second direction, the second direction being in an opposite rotational sense to the first direction. The first and second edges of each of the one or more elongated members meet at an apex.

In certain embodiments, a transverse cross-section of the needle body has a perimeter and the one or more elongated members lie entirely on the perimeter when the needle is viewed in cross-section along the longitudinal axis. In certain other embodiments, the first and second edges of each of the one or more elongated members curve inwards from the perimeter and meet at an apex. In still other embodiments, the apices of the respective two or more, such as two, elongated members meet at the centrally disposed longitudinal axis. In another embodiment, there is just one elongated member whose apex lies on the longitudinal axis.

Typically the needle is such that the first edge has a bevel on its surface interior to the needle, and the second edge has a bevel on its surface exterior to the needle. This means that in certain embodiments the first edge is a leading edge, and the second edge is a trailing edge, during the cutting action as the needle is rotated about its longitudinal axis.

Typically, the needle body is cylindrical, in which case a transverse cross-section of the body has a circumference and the one or more elongated members protrude from an arc of the circumference. In some embodiments, the arc has an angular extent between π/8 (“Pi/8”) and π (“Pi”) radians, such as π/4 (“Pi/4”), π/6 (“Pi/6”) or π/3 (“Pi/3”), of the circumference.

In other embodiments, the one or more, such as four, elongate members are mounted flexibly at their respective points of contact with the cylindrical tube, and wherein the first and second edges of each elongate member are contiguous with one another, such as form together an arc of a circle or an oval. In such embodiments, the elongate members flare outwards from a centrally disposed longitudinal axis as the needle is inserted into a subject, but the elongate members revert to their respective original positions as the needle is withdrawn from the subject. In certain such embodiments, the one or more elongate members are hinged or sprung at their respective points of contact with the cylindrical tube, thereby providing flexibility.

FIGS. 6A-6N illustrate cross-sectional and perspective views of the distal ends of various exemplary embodiments of a needle for carrying out a rotational biopsy as further described herein. Needle 600, shown in other illustrations of this application, may refer to any of needles 610, 620, 630, 640, or 650, as well as to any other needle useable with this device. Edges, e.g., sharp or beveled edges, on needle 600 allow for rotational cutting action. Sharp distal tips on the beveled needle 600 also facilitate distal advancement of said needles into tissue to reach the locus of interest.

Referring to FIG. 6A, needle 610 includes a tubular body 615 and a sharpened distal end 611 and a lateral opening located proximal to the distal end, leading to a hollow lumen. Needle 610 also defines longitudinal axis 601. Leading edge 612 and trailing edge 613 curve around and extend along the perimeter of needle body 615 (i.e., they do not bend inward towards axis 601.) As used herein the terms “leading edge” means the edge leading towards the distal tip that cuts tissue as the needle rotates. The term “trailing edge” is the other edge, i.e., the edge that follows the leading edge when the needle rotates as above. Note that the trailing edge may also cut tissue. FIG. 6B is a view of needle 610 looking proximally from the distal end of the longitudinal axis. Note that leading edge 612 and trailing edge 613 lead along the same perimeter as needle body 615. When inserted into the body and rotated about the longitudinal axis, the leading and trailing edges 612, 613 will cut surrounding tissue. FIG. 6C shows a perspective view of needle 610. Needle 610 may be beveled, and may be beveled such that leading edge 612 is beveled on the inside (e.g. the side nearer the lumen of the needle), and trailing edge 613 is beveled on the outside. This beveling arrangement promotes the movement of cut tissue toward the lumen of the needle.

FIG. 6D shows an alternative embodiment, needle 620. As in the embodiment of FIG. 6A, needle 620 has a tubular body 625 which defines longitudinal axis 601 and includes a sharpened distal end 621 and a lateral opening located proximal to the distal end, leading to a hollow lumen. The outer edge of the needle may curve inward at distal end 621, toward longitudinal axis 601. FIG. 6E shows a view of needle 620 looking proximally from the distal end of the longitudinal axis. In this view, it can be seen that leading edge 622 and trailing edge 623 bend inwards toward axis 601. Distal end 621 may end at axis 601, or at any point between the circumference of needle body 625 and axis 601. Also shown in FIG. 6E, the curvatures of leading edge 622 and trailing edge 623 may be different from one another, (i.e., the portion leading to distal end 621 is asymmetric in this view, i.e. asymmetric about a mirror plane including axis 601 and tip 621). Needle 620 may also have symmetric leading portions, i.e., symmetric in such a mirror plane. Similarly, the leading portion may curve or twist in a clockwise or counterclockwise direction around axis 601 as it extends towards distal end 621. When inserted into the body and rotated about the longitudinal axis 601, the leading and trailing edges 622, 623 can cut the surrounding tissue. FIG. 6F shows needle 620 in a perspective view. Leading end 622 and trailing end 623 may be beveled, as described above.

Although the distal tip of the needle, as shown in the embodiments of FIGS. 6A-6F, appears blunt, it is typical for the curvature on the most distal point to be shallower than depicted in the figures so that the distal tip of the needle is a sharp point.

FIG. 6G shows an alternative embodiment, needle 630. Needle 630 defines longitudinal axis 601 through needle body 635. In this embodiment, two regions extend toward, and, optionally, converge at, distal end 631. A lateral opening is located proximal to the distal end, and leads to a hollow lumen. Needle 630 includes first and second leading edges 632a and 632b, respectively, and first and second trailing edges 633a and 633b, respectively. FIG. 6H shows a view of needle 630 looking proximally down the longitudinal axis from a distal position. Here, it can be seen that curvature of leading edges 632a-b is the same as those of trailing edges 633a-b (i.e. portions leading to distal end 621 are symmetric in this view, i.e. symmetric about a mirror plane including axis 601). Needle 630 may also have asymmetric leading portions, and each such portion may itself be asymmetric, as described for FIG. 6E. Similarly, these leading portions may curve or twist in a clockwise or counterclockwise direction around axis 601 as they extend towards distal end 631. When inserted into the body and rotated about the longitudinal axis, the leading and trailing edges 632a, 632b, 633a, 633b can cut surrounding tissue. As above, leading edges 632a-b may be beveled on the inside, and trailing edges 633a-b may be beveled on the outside, e.g., to direct cut tissue towards the lumen of the needle.

FIG. 6J shows an alternative embodiment, needle 640. Needle 640 defines longitudinal axis 601 through needle body 645. In this embodiment, multiple regions 641, e.g., three, four, five, or six, are located at the distal end of the needle 640 and extend slightly inwardly. Regions 641 include sharp leading edges 642. Further, the inner surface 643 of each region 641 can be beveled. FIG. 6K shows a view of needle 640 looking proximally down the longitudinal axis from a distal location. Here, it can be seen that regions 641 extend slightly inward toward a hollow lumen (not drawn to scale for illustrative purposes). Such needle tip may be fabricated from a shaped memory alloy such as nitinol (NiTi). When needle 640 is advanced into tissue, the sharp edges 642 can cut tissue. Further, as shown in FIG. 6L, if the needle 640 is both advanced distally and rotated around axis 601, the accumulation of tissue within the lumen can create an outward force on regions 641, causing the regions 641 to move outward. Once tissue sampling is complete, the construct of region 641 again extends slightly inward and because the inner surfaces 643 are beveled, the cut tissue can be trapped within the lumen as the needle 640 is removed. FIG. 6M shows a top-down view of needle 640 in such a position. When rotation of the needle 640 is completed, the structural force of regions 641 overcomes that of the entrapped tissue, resulting in return of regions 641 to their original position (as shown in FIG. 6K), thereby preventing tissue inside the lumen from being lost as the device is removed from the patient. In another embodiment, a vacuum apparatus may be attached proximally (not shown) thus allowing immediate retrieval of the specimen while the device remains in the patient.

An example of another embodiment of a needle having a single elongated member is needle 650 shown in FIG. 6N. It can be seen that such an embodiment can be obtained from a standard needle, as used in medicine and surgery, by cutting away portion at one side 653 of the distal tip, thereby creating a beveled edge. If the edge so created is beveled in the opposite sense to the beveling at the opposite edge 652, the rotational cutting efficacy of the needle is enhanced and contrasts with the beveling of a standard needle, which is in the same sense along all edges. In this embodiment, distal tip 651 is flush with the perimeter of the body 655 of the needle when viewed down a longitudinal axis 659.

For reference, when needle 600 (which includes any of needles 610, 620, 630, 640 and 650) is attached to a biopsy device such as biopsy device 100a by luer or other connection, the longitudinal axis of the needle (e.g. axis 601) will typically be collinear with longitudinal axis 301. See, e.g. FIG. 1. As noted herein, vacuum may be applied to the lumen of the needle so that cut tissue may be removed by aspiration.

The geometries and orientations of needles 610, 620, 630, 640, and 650 illustrated in FIGS. 6A-N are exemplary, and it is understood that certain modifications may be made, including the following. The distal ends of the needles may be modified (e.g., by sharpening, pointing, etc.) to promote advancement of the needle into tissue. Referring to the examples illustrated in FIGS. 6A-F, more than one leading and trailing edges (i.e. more than one portion leading to a distal end) may be present on a single needle. Similarly, referring to FIGS. 6G-I, more than two leading and trailing edges (i.e., more than two portions leading to a distal end) may be present on a single needle. Curving or twisting portions leading to the distal end may curve or twist in a clockwise or counterclockwise direction. Leading and trailing edges may be straight or curved. Curvature of the leading and trailing edges may be the same (i.e., symmetric about a mirror plane including axis 601) or different (i.e. asymmetric about a mirror plane including axis 601), and may be concave (as illustrated) or convex. The distal end or ends may lie at the same circumference as that of the needle casing, may curve inwards and lie on axis 601, or may lie on any point or points in between. Referring to the examples illustrated in FIGS. 6J-M, sharpened leading edges 642 may be shaped with a saw tooth or irregularly and/or asymmetrically sharp configurations. Other modifications may also be made consistent with the teachings of this disclosure.

Needle 600 (which includes any of needles 610, 620, 630, and 640) may be used for any type of biopsy or similar technique, including fine biopsy, including, without limitation, biopsy of the thyroid, salivary gland, lung, pleura, liver, spleen, kidney, pancreas, adrenal, lymph node, breast, prostate, muscle, brain, intestine and any neoplastic solid mass, and core biopsy, including, without limitation, biopsy of the thyroid, salivary gland, liver, spleen, kidney, pancreas, adrenal, lymph nodes, breast, prostate, muscle, brain, intestine and any neoplastic intravisceral or extravisceral solid mass. Examples include needles of 22 to 27 gauge for fine biopsy, and 8 to 14 gauge for core biopsy. Such needles may be made from materials used to make conventional biopsy needles, including, without limitation, stainless steel, various alloys such as nickel titanium and certain high tensile strength plastics, such as polycarbonate, and carbon composites.

### Operation

Operation of the aspiration biopsy device 100a is illustrated by FIGS. 7A-B. First, rotation of hub 200 is described. Referring to FIG. 7A (and as described elsewhere herein) hub 200 is disposed in casing 300a, and slide 208 is disposed within receiving chamber 315. Handle 220 is connected to stem 221, which slidably inserts through distal slot 340. Stem 221 terminates at end 222, which engages helical groove 209. FIG. 7A shows control 220 in a fully retracted position, that is, control 220 is as far in the proximal direction as possible. A user may manually (e.g. by using a thumb or finger) extend handle 220 in the distal direction indicated by arrow 701. During such extension, the movement of end 222 engaging helical groove 209 causes hub 200 to rotate around axis 301 relative to casing 300a, as shown by arrow 702.

FIG. 7B shows needle biopsy device 100a when control 220 has been moved partway along the slot in the manner described above. A user may now continue to move the control 220 in the distal direction, or may retract it by pulling it back in the proximal direction as indicated by arrow 703. Retraction of the control in this manner will cause hub 200 to rotate in the opposite direction as it did during the distal movement, as indicated by arrow 704.

When a needle (not shown here) is attached to the hub, the needle will also rotate. By repeatedly moving the control 220 in the distal and proximal directions, a user may cut tissue for biopsy with fine control.

In certain implementations, the distance between full extension and retraction of control 220 may be from about 0.5 cm to about 2.0 cm. In other implementations, the device may be dimensioned and oriented for other distances. This distance will be determined in part by the length and orientation of helical groove 209 and slot 340.

FIGS. 7A-B also illustrate operation of diaphragm assembly 400. Diaphragm assembly 400, including partition 410 disposed in the distal portion of second chamber 350, may be slidably moved in the proximal and distal directions relative to the second chamber. FIG. 7A shows diaphragm assembly 400 in a fully extended position, that is, disc 410 is as far in the distal direction as possible, stopped by distal end-stops 351. Distal position of disc 410 effects compression of optional spring 122. As such, spring 122, between casing 300a is fully compressed between casing 300a and diaphragm assembly 400. Diaphragm assembly 400 may be retracted in the proximal direction indicated by arrow 705 by moving handle 420, e.g. by a thumb or other finger. Such retraction proximally of diaphragm 410 will create a reduced pressure, such as a partial vacuum, in the distal portion of second chamber 350. The distal portion of the second chamber 350 is in fluid communication with central barrel 310, which is in fluid communication with the lumen of the hub 210. When a needle is attached, hub chamber 210 is in fluid communication with the lumen of the needle. Thus, retraction of handle 420 will create a reduced pressure, such as a partial vacuum, in the lumen of the needle, allowing the user to remove cut tissue by aspiration.

FIG. 7B shows biopsy device 100a when diaphragm assembly 400 has been partially retracted in the manner described elsewhere herein. Also, diaphragm assembly 400 is at an intermediate stopping point, since bar 414 (not shown in this view) on flexible rod 412 has engaged an intermediate stop 365 (see FIGS. 7C-D). Spring 122 is thus partially expanded. A user may now continue to move diaphragm assembly 400 by withdrawing handle 420 away from casing 300a. Handle 420 is connected to diaphragm 410 by flexible rod 412. Other manners of controlling motion of partition 410 are consistent with the devices herein. Depression of handle 420 disengages a lateral member 414 from stops 365. The release of this locking mechanism allows optional spring 122 to release tension, thereby abetting the proximal motion of diaphragm 410. The user may then continue to retract diaphragm assembly 400, or may extend it in the distal direction by pushing handle 420 in the distal direction, as indicated by arrow 705. When handle 420 is extended in the distal direction, positive pressure will be generated in the distal portion of the second chamber 350 and, correspondingly, in the lumen of the attached needle. Such positive pressure may be used to extrude the sample for analysis, after the needle has been withdrawn from the subject. The optional spring 122 serves to resist rapid emptying and prevent sudden undesirable extrusion of all tissue from the vacuum chamber 310 and needle at once. As partition 410 is moved in the distal direction, negative pressure may be generated in the proximal portion of second chamber 360. As described above, such pressure can be minimized by vent 361, which fluidly communicates venting chamber 360 with the surrounding atmosphere.

FIGS. 7C and 7D illustrate a ratcheting mechanism of diaphragm assembly 400. In these expanded transverse cross-sectional views, bar 414 is seen to extend from flexible rod 412. In FIG. 7C, bar 414 engages intermediate stop 365, preventing the diaphragm assembly from being drawn in the proximal or distal direction. FIG. 7D illustrates that by pushing handle 420 towards casing 300a, flexible rod 412 will depress, disengaging bar 414 from intermediate stop 365.

Once a sample has been extruded, needle 600 (which includes any of needles 610, 620, 630, and 640) may be removed from hub 200. The device may be cleaned, and any residual tissue or liquids may be removed, by injecting cleaning liquid or air into the device using a syringe attached at proximal end, once cap 500 is removed.

Advantageously, the rotational cutting method using the needle and/or the device described herein can prevent sampling of adjacent non-target tissue such that tissue sampled is precisely from the locus of interest. For example, using ultrasound or some other visualization tool for guidance, the tip of the biopsy needle may be advanced to the locus of interest within a lesion such as a thyroid lesion. Traditional fine needle aspiration methodology employs a to-fro motion on the needle, often with simultaneous suction provided by an attached syringe. The acquisition of a sample in this manner may include irrelevant material outside the locus of interest. This can be minimized using the biopsy device with a rotational needle as described herein, whereby the aspiration occurs using rotary cutting of the needle tip without the need for proximal or distal translation.

Additionally, in some embodiments, a solid stylet may be advanced to the leading edge of needle tip 600 (which includes any of needles 610, 620, 630, 640, and 650) through proximal end hole in lieu of cap 500. Having such a stylet in place prevents, or substantially hinders, the accumulation of unwanted non-target tissue within the needle lumen as the assembly is advanced in the body to the locus of interest. Once the tip of the needle has reached the locus (as defined by imaging with or without robotic instrumentation), the stylet can be removed and the sample obtained as described herein.

Additionally, the device described herein, with a sharp distal needle tip, may be utilized in some embodiments for core biopsy in which larger gauge needles utilize rotational cutting while forward translation of the needle assembly is additionally effected by manual or mechanical means. This may be particularly useful in the setting of robotic assisted biopsy, where the locus of interest has been determined three dimensionally from imaging data. It is proposed that cutting tissue simultaneously using rotary as well as forward translational movement may allow for a greater degree of cutting efficiency and exactitude when compared with other biopsy methods that use needle translation only without simultaneous rotary cutting action. When such core specimens are obtained, they may be left within needle lumen for later retrieval (after the device has been removed from the patient) or such specimens may be instantaneously retrieved by the application of a vacuum or mechanical suction device peripheral to the biopsy device.

FIGS. 8A-B illustrate alternative embodiments of a biopsy device 100b with needle 600 attached to hub 200 (e.g., by a luer connection). As shown in FIG. 8A, casing 300b assumes an ergonomic shape, being dimensioned to feel comfortable in the hand of a user. Casing 300b may include one or more finger grooves or indentations 309 that may be shaped to accept one or more fingers and thumb. Also shown in this embodiment, the diameter of central chamber 330 may be the same or about the same as that of barrel 310. FIG. 8B shows a transverse cross-sectional view of a proximal region of device 100b. In this view, it is seen that control 220 and handle 420 may be angularly offset from one another with respect to longitudinal axis 301. The degree of offset may be arbitrary and may be altered to maximize comfort and usability of the device. Other aspects of the exemplary embodiment depicted in FIGS. 8A-B correspond to those of other embodiments described herein.

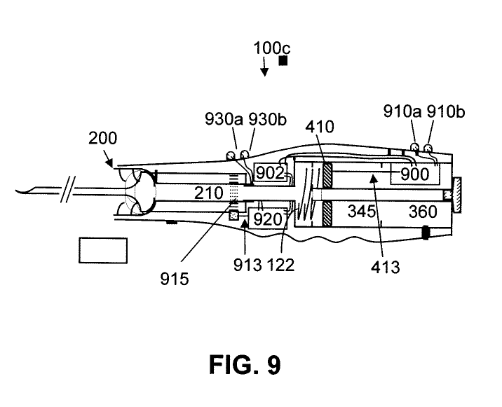

FIG. 9 illustrates alternative embodiments of a device wherein a vacuum or partial vacuum is created by use of a motor rather than by manual movement of a handle. As shown, biopsy device 100c includes motor 900, powered by power source 902. Motor 900 moves rod 413. Rod 413 moves disc 410 in the proximal direction to create a vacuum or partial vacuum in the distal portion of second chamber 350. Rod 413 may also be moved in the proximal direction to create positive pressure to eject a tissue sample. Motor 900 can be controlled by one or more buttons, e.g., buttons 910a and 910b, for turning the motor on and off, and controlling the direction of movement of disc 410. This embodiment may be constructed with or without spring 122.

In an additional embodiment, a biopsy device such as biopsy device 100c shown in FIG. 9, may be equipped with a motor to rotate hub 200. FIG. 9 shows motor 920 (which may also be powered by power source 902) which may drive shaft 913, connected to gear 915. Gear 915 may engage teeth on needle mount hub 200 so that rotation of gear 915 may rotate needle mount hub 200. Motor 920 may be controlled by one or more buttons, e.g., buttons 930a and 930b.

Other known techniques and materials may be used to rotate hub 200 and move disc 410 by motor. Also, while FIG. 9 shows both the hub and the disc moved by a motor, such motorized movements are independent, and can be present in separate implementations, with an alternative being manual movement of both as described elsewhere herein.

### Materials

In certain embodiments, components of biopsy device 100a, 100b, or 100c (including, but not limited to casing 300a-c and hub 200) may be made of a suitable plastic material (e.g., polycarbonate). In some embodiments, casing 300a-c may have solid portions and hollow portions, e.g., in order to reduce weight or cost of materials. Also, as described elsewhere herein, the part of casing 300a-c surrounding vacuum chamber 350 and venting chamber 360 may be hollow to accommodate part of diaphragm assembly 400. In certain implementations, various components may be transparent, e.g., to enable the user to visualize fluid, tissue, instrumentation, etc., within the device. In other implementations, they may be colored and either opaque or transparent.

End 222 may be constructed to reduce friction as it engages helical groove 209, and to facilitate linear movement of control 220 and corresponding rotation of hub 200. For example, in implementations, end 222 may be rounded or ball-shaped. Stem 221 and/or end 222 may be made from a suitable plastic or polymer (e.g. polycarbonate) and/or coated with Teflon, silicone, or a similar material with low coefficient of friction. In other implementations, end 222 may be a metallic ball bearing, similar to the end of a conventional ballpoint pen. Other constructions and materials of stem 221 and end 222 can easily be envisaged.

The gripping surfaces of the device (e.g. outer surfaces of casing 300a-c) can be covered with a thermoplastic elastomer, including those under trade names Megol, Santoprene and Multibase, as well as silicone elastomers. The casing may comprise a unitarily moulded skeleton onto which the elastomer areas are moulded in a separate injection moulding step (see, e.g. International Application PCT/US 1999/020606 of Volpenhein et al.). The points of contact between the stationary and active portions of the device may be coated with Teflon, silicone, or a similar material with low coefficient of friction.

Disc 410 and end cap 500 may be made from lightweight plastic, and, for the airtight seals to vacuum chamber 350 and central chamber 330, disc 410 may be made of lightweight plastic constructed with a peripheral rim of rubber or a deformable resilient plastic such as a silicone.

Control 220 and handle 420 may, in certain embodiments, pivot laterally for better ergonomic control. These items may be ridged, so as to facilitate grip, and composed of an elastomeric compound bound to lightweight durable plastic.

In addition, larger gauge embodiments for core biopsy, although not shown, are envisioned. In such embodiments, the entire assembly may be advanced using manual or mechanical methods (including spring, hydraulic, pneumatic and motorized methods) for translation. In addition, computer assisted robotic methods of controlling a biopsy performed by the device and/or needle herein may be envisaged.

A number of embodiments have been described. Nevertheless, it will be understood that any single device may include features of the particular devices illustrated, and various modifications may be made, including modifications to shape, size, and arrangement of parts, without departing from the spirit and scope of the disclosure, including the aforementioned nonvascular applications and others. Accordingly, other embodiments are within the scope of the following claims.

What is claimed is:

1. A biopsy device configured to perform a biopsy by rotating a needle.

2. A biopsy device comprising
a casing having a proximal end and a distal end, and defining a longitudinal axis running between the proximal and distal ends;
a first chamber disposed within the casing and opening to the distal end;
a second chamber disposed within the casing between the first chamber and the proximal end;
a hub disposed coaxially within the first chamber and including a feature on its external surface;
a needle assembly mounted on and disposed coaxially with the hub, the needle having a lumen, the lumen in fluid connection with the second chamber; and
a control configured to engage the feature on the external surface of the hub, and to cause a rotation of the hub and needle assembly about the longitudinal axis.

3. The biopsy device of claim 2, wherein the feature comprises a helical groove, and wherein the control comprises a pin disposed in a longitudinally-aligned slot in the casing, the pin engaging the helical groove, and wherein displacement of the pin in the slot in a direction parallel to the longitudinal axis effects a rotation of the hub and the needle assembly about the longitudinal axis.

4. The biopsy device of claim 3 wherein the pin is attached to a handle that may be moved manually by an operator of the device, thereby causing the longitudinal motion of the pin.

5. The biopsy device of claim 3 wherein a displacement of the pin from a proximal location to a distal location effects a rotation of the hub and needle assembly through more than one complete rotation about the longitudinal axis.

6. The biopsy device of claim 2 wherein the needle assembly is reversibly attached to the hub.

7. The biopsy device of claim 2 wherein the needle comprises a distal tip and wherein the lumen has a distal opening proximal to the distal tip.

8. The biopsy device of claim 7 wherein the needle is configured to cut tissue at the biopsy site in directions transverse to the longitudinal axis, when rotated about the longitudinal axis.

9. The biopsy device of claim 2 wherein the second chamber contains a vacuum or a partial vacuum.

10. The biopsy device of claim 9 further comprising a partition disposed in the second chamber, and dividing the second chamber into a proximal portion and a distal portion.

11. The biopsy device of claim 10, wherein the partition is connected to a control member, wherein displacement of the control member in the distal and proximal directions effects a corresponding displacement of the partition, resulting in greater or reduced pressure respectively in the distal portion of the second chamber.

12. The biopsy device of claim 11 further comprising a vent fluidly communicating the proximal portion of the second chamber with atmosphere external to the device.

13. The biopsy device of claim 12 further comprising a ratchet to control movement of the partition within the vacuum chamber.

14. The biopsy device of claim 10 wherein the partition is a disc.

15. The biopsy device of claim 2 wherein the casing and hub comprise molded plastic.

16. The biopsy device of claim 15 wherein the molded plastic is transparent.

17. The biopsy device of claim 2 wherein the casing assumes an ergonomic shape, and wherein the distal end of the casing is narrower in transverse cross-section than the proximal end of the casing.

18. A method of performing a biopsy of a tissue, the method comprising:
controlling the device of claim 1 so that the needle is inserted into the tissue;
rotating the needle about the longitudinal axis of the device, thereby cutting tissue;
reducing pressure in the lumen; and
withdrawing the needle from the tissue, thereby removing a portion of cut tissue.

19. The method of claim 18 wherein the biopsy is a fine biopsy or a core biopsy.

20. The method of claim 18 wherein the device is operated using one hand of an operator.

21. A biopsy needle comprising:
a tubular body having a centrally disposed longitudinal axis running between distal and proximal ends of the body, the body further comprising a lumen disposed along the longitudinal axis, and a distal opening; and
one or more elongated members extending distally from the distal opening and contiguous with the tubular body, each of the one or more elongated members having a first edge and an opposed second edge, the first edge configured to perform a cutting action as the needle is rotated about the longitudinal axis in a first direction, the second edge optionally configured to perform a cutting action as the needle is rotated about the longitudinal axis in a second direction, the second direction being in an opposite rotational sense to the first direction.

22. The needle of claim 21 wherein the first and second edges of each of the one or more elongated members meet at an apex.

23. The needle of claim 22, having one or two elongated members.

24. The needle of claim 21, wherein a transverse cross-section of the body has a perimeter and the one or more elongated members lie entirely on the perimeter when the needle is viewed in cross-section along the longitudinal axis.

25. The needle of claim 21, wherein a transverse cross-section of the body has a perimeter and wherein the first and second edges of each of the one or more elongated members curve inwards from the perimeter and meet at an apex.

26. The needle of claim 25 wherein the apices of two or more elongated members meet at the longitudinal axis.

27. The needle of claim 25, having two elongated members, and wherein the apices of the two elongated members meet at the longitudinal axis.

28. The needle of claim 25, having a single elongated member, and wherein the apex lies on the longitudinal axis.

29. The needle of claim 21 wherein the first edge has a bevel on its surface interior to the needle, and the second edge has a bevel on its surface exterior to the needle.

30. The needle of claim 29, wherein the first edge is a leading edge, and the second edge is a trailing edge, during the cutting action as the needle is rotated about its longitudinal axis.

31. The needle of claim 21, wherein the body is cylindrical wherein a transverse cross-section of the body has a circumference and the one or more elongated members protrude from an arc of the circumference, wherein the arc has an angular extent between π/8 and π of the circumference.

32. The needle of claim 21, wherein the one or more elongate members are mounted flexibly at their respective points of contact with the cylindrical tube, and wherein the first and second edges of each elongate member are contiguous with one another.

33. The needle of claim 32, comprising four elongate members.

34. The needle of claim 32, wherein the elongate members flare outwards from the longitudinal axis as the needle is inserted into a subject, and wherein the elongate members revert to their respective original positions as the needle is withdrawn from the subject.

35. The needle of claim 32, wherein the one or more elongate members are hinged or sprung at their respective points of contact with the cylindrical tube.

36. A biopsy device comprising:
a proximal casing having a longitudinal axis; and
a distal needle according to claim 21 affixed to the casing, and configured to rotate about the longitudinal axis.
